(19) United States
(12) Patent Application Publication
(10) Pub. No.: US 2026/0188479 A1
(43) Pub. Date: Jul. 2, 2026

(54) USER RECOGNITION IN A RPM SYSTEM

(71) Applicant: Withings, ISSY LES MOULINEAUX (FR)

(72) Inventors: Samuel ATTIA, ISSY LES MOULINEAUX (FR); Vincent HOULBRÈQUE, ISSY LES MOULINEAUX (FR)

(21) Appl. No.: 19/425,517

(22) Filed: Dec. 18, 2025

(30) Foreign Application Priority Data
Dec. 26, 2024 (FR) FR2415305

(51) Int. Cl. G16H 40/63

(52) U.S. Cl. CPC G16H 40/63

(57) ABSTRACT

A system includes a physiological measurement device (the device) and a server with a user profile, wherein the device generates physiological data and recognition data, and the recognition data are used to compute a recognition score to determine whether the person using the device is the user of the user profile or not. Based on the recognition score, actions are taken.

### CROSS-REFERENCE TO RELATED APPLICATIONS

This application claims priority to French Patent Application No. 2415305, filed December 26, 2024, the entire content of which is incorporated herein by reference in its entirety.

### FIELD

The invention relates to the field of recognizing a user performing health-related measurements by a physiological measurement device and, more precisely, the attribution of those health-related measurements to a specific user. This recognition is useful in the field of remote patient monitoring (RPM) and RPM systems.

### BACKGROUND

Electronic body scales usually use weight to recognize a user among a plurality of user profiles. This recognition is usually carried out at the beginning of a measurement session. Either the user is recognized, and the measured data are attributed to the user (on the device and on a remote device, such as a phone or a server), or the user is not recognized, and the measured data is not attributed to the user. Most of the time, a guest mode is implemented so that a registered user may still access the non-attributed data.

However, with the development of remote patient monitoring (RPM) and cellular devices, the user has fewer and fewer control of his or her own devices and the data management flow. For example, with a cellular body scale, which is pre-configured for a specific user, all data coming from that scale may automatically be attributed to the user. This may cause data belonging to someone else being attributed to the user and thus undermining the efficiency of the RPM

### SUMMARY

An aspect of the invention is to provide a server, a RPM system, and methods associated therewhith which does not present the limitations mentioned previously.

In an aspect, the disclosure relates to a server storing a user profile associated with a user, and configured to exchange data with a physiological measurement device. The physiological measurement device may be configured so that every data generated by the physiological measurement device is systematically associated with the user profile of the server.

The server is configured to receive from the physiological measurement device physiological data generated by the physiological measurement device, wherein at the server the physiological data are associated with the user profile. The server is further configured to receive from the physiological measurement device recognition data associated with the physiological data. The recognition data are data used to determine whether the physiological data were generated by the user of the user profile.

Based at least on the recognition data, the server is configured to compute a recognition score based. This recognition score is then used to manage the physiological data.

For example, managing may include attaching the recognition score to the physiological data so that a remote monitoring interface may visually flag the physiological data or so that another server may delete the physiological data (especially when the recognition score reflects that the physiological data were not generated by the user of the user profile). Managing may include removing the physiological data from the user profile (especially when the recognition score reflects that the physiological data were not generated by the user of the user profile). Managing may include triggering a user request to confirm or reject the physiological data (especially when the recognition score reflects that there is a doubt about the person who generated the physiological data). Managing may include generating instructions to delete the physiological data of the user profile based on the recognition score, either at another server or at the physiological measurement device.

The recognition data may be taken from the physiological data. In an embodiment, the physiological data is a user weight, and the recognition data are the user weight as well.

According to this aspect, a system is also disclosed, wherein the system is notably a RPM system, and includes the server as presented above and a physiological measurement device (the device). The server and the device are configured to exchange data through a communication network.

Particularly, the device is a mono-user device. This means that the device is configured so that every data generated by the device is systematically associated with the user profile of the server.

The additional features provide further implementation details.

In an embodiment, the user profile includes reference data, and the recognition score is computed based on a comparison between the reference data and the recognition data.

In an embodiment, computing the recognition score takes into account: a value difference between a value of the generated recognition data and a value of the reference data, and a time difference between a timestamp of the generated recognition data and a timestamp of the reference data. This allows taking into account the variation of physiological data over time.

In an embodiment, a reference score is attached to the reference data and computing the recognition score takes into account the reference score, so that the recognition score can only be strictly lower than the reference score.

In an embodiment, the server or system is configured to, in response to determining that the recognition score is above a certainty threshold, update the reference data with the generated recognition data. The reference data are then up-to-date, which allows a recognition process as good as it can be.

In an embodiment, the system is, further configured to, in response to determine that the recognition score is below an uncertainty threshold, remove the generated physiological data and the generated recognition data from the user profile.

In an embodiment, the user profile includes a notification channel to the user and the system is further configured to, in response to determining that the recognition score is below the certainty threshold, trigger by the server a request on the notification channel to confirm or reject that the received physiological data and the recognition data are to be attributed to the user profile.

The server, then, in response to receiving the confirmation, updates the reference data of the user profile with the generated recognition data, and/or in response to receiving the rejection, removes the generated recognition data and the generated physiological data of the user profile.

In an embodiment, the server is further configured to, in response to determining that the recognition score is below a certainty threshold and above an uncertainty threshold and in the absence of receiving a confirmation or a rejection within a predetermined time period, updating the reference data with the obtained recognition data, or/and in response to determining that the recognition score is below the uncertainty threshold and in the absence of receiving a confirmation or a rejection within a predetermined time period, deleting the recognition data and the physiological data of the user profile.

In an embodiment, the server includes a first server and a second server, and the user profile includes a first user profile stored at the first server and a second user profile at the second server, wherein the second user profile includes at least the first user profile, wherein the second user profile and the first user profile are associated with the same user, and the second server is connected to a remote monitoring interface. The system is further configured to send by the first server the physiological data with the recognition score to the second server, and remove the generated physiological data of the second user profile based on the recognition score.

In particular, determining and removing are performed by the second server, and the removing is done on the second user profile. The notification channel is included in the second user profile, and triggering is performed by the second server.

In an embodiment, the system is, further configured to send to the physiological measurement device instructions to delete the generated physiological data and the generated recognition data.

The notification channel may involve a user terminal, such a user mobile phone. In an embodiment, the user cannot manage the physiological data from his or her user terminal. This avoids deleting or data alteration by the user.

In an embodiment, the system further comprises initializing the reference data of the user profile, wherein initializing is done by: - sending through a notification channel a request to use the physiological measurement device during a time window and receiving recognition data during that time window, and setting the reference data as the recognition data, - initially providing the reference data by a third party, - manually inputting the reference data.

In an embodiment, the system further comprises a remote monitoring interface, wherein the remote monitoring interface has a specific visual feature for the physiological data with a recognition score under a certainty threshold.

In an embodiment, the recognition data are taken from the physiological data. This allows to reduce the effort and constraint on the user. Alternatively, the recognition data are different from the physiological data. This allows to have recognition data better adapted for recognition than physiological data, notably in the case in which the physiological data are not appropriate to recognize a user.

In an embodiment, the recognition data include at least one of weight data and impedance data.

According to this aspect, the disclosure also relates to a method for remotely monitor patients, using a system as disclosed above, wherein the method comprises: generating physiological data by the physiological measurement device, generating recognition data by the physiological measurement device, wherein the recognition data are associated with the physiological data, - receiving by the server the generated recognition data and the generated physiological data, wherein the physiological data and the recognition data are attributed to the user profile of the server, - computing a recognition score based at least one the generated recognition data, the recognition data being used to determine whether the physiological data were generated by the user of the user profile, - managing the generated physiological data based at least one the recognition score.

According to another aspect, the disclosure also relates to a physiological measurement device (the device) storing a user profile associated with a user, and configured to exchange data with a server. The device may be configured so that every data generated by the device is systematically associated with the user profile of the device and thus of the server.

The device is configured to generate physiological data, wherein at the server the physiological data are associated with the user profile, and to generate recognition data associated with the physiological data. The recognition data are data used to determine whether the physiological data were generated by the user of the user profile.

Based at least on the recognition data, the device is configured to compute a recognition score based. The recognition score may be attached to the physiological data. The recognition score and the physiological data are then sent to a server. This recognition score is then used to manage the physiological data, at the device, and/or at a server.

Managing the physiological data may be as disclosed previously.

The device may notably be a body scale.

This other aspect also relates to a method for remotely monitoring a patient using the device previously disclosed.

In an embodiment, there is provided a non-transitory computer-readable medium storing instructions which, when executed by one or more processors of a server of a remote patient monitoring, RPM, system, cause the server to:

receive, from a physiological measurement device, generated physiological data and generated recognition data associated with a user profile stored at the server,

compute a recognition score based at least on the generated recognition data and reference data stored in the user profile, the recognition score being representative of a likelihood that a user associated with the user profile generated the physiological data,

compare the recognition score to at least one threshold including a certainty threshold and optionally an uncertainty threshold,

when the recognition score is above the certainty threshold, maintain the physiological data as attributed to the user profile and update the reference data with the generated recognition data,

when the recognition score is below the uncertainty threshold, remove at least the generated physiological data from the user profile and optionally send an instruction to the physiological measurement device to delete the generated physiological data and the generated recognition data, and

when the recognition score is between the certainty threshold and the uncertainty threshold, trigger, via a notification channel associated with the user profile, a user request to confirm or reject that the physiological data and the recognition data are to be attributed to the user profile, and process a received confirmation or rejection to update or remove the physiological data and the recognition data from the user profile.

In an embodiment, the instructions further cause the server to provide, to a remote monitoring interface, only physiological data that has been confirmed or inferred as originating from the user based on the recognition score, such that the physiological data presented on the remote monitoring interface is usable by a healthcare professional to support a diagnostic assessment.

The additional features provided above are also applicable inasmuch as they relate to the server, except that the recognition score is not computed by the server.

### BRIEF DESCRIPTION OF THE DRAWINGS

Other features will be visible on the following non limitative drawings:

FIG. 1 shows a remote patient monitoring, RPM, system, according to an embodiment of the disclosure,

FIG. 2 shows a device (a body scale)according to an embodiment of the disclosure,

FIG. 3 shows a remote monitoring interface run on a monitoring device according to an embodiment of the disclosure,

FIG. 4 shows two different server configurations according to variants of the disclosure,

FIG. 5 shows physiological data (user weight data) and recognition data (user weight data as well) taken at different times and with different values,

FIG. 6 shows two examples for computing a recognition score,

FIG. 7 shows an example of a display mode with notification levels on the remote monitoring interface according to an embodiment of the disclosure,

FIG. 8 shows an example of a user request via the notification channel, according to an embodiment of the disclosure,

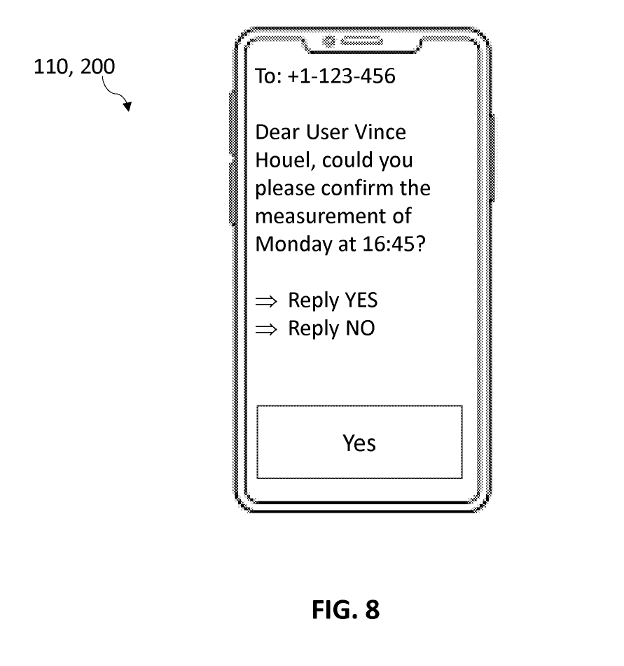

FIG. 9 shows a RPM method performed by a RPM system, according to an embodiment of the disclosure.

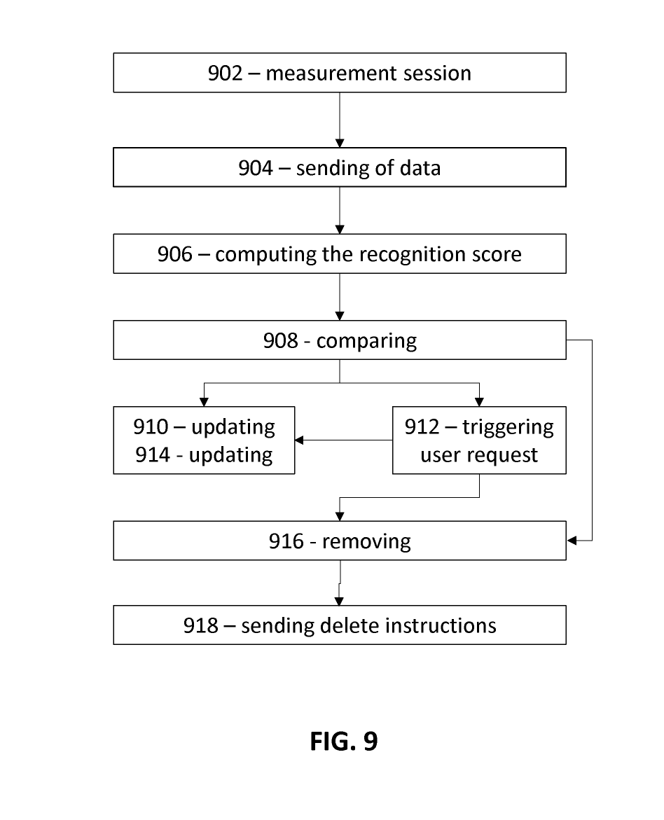

### DETAILED DESCRIPTION

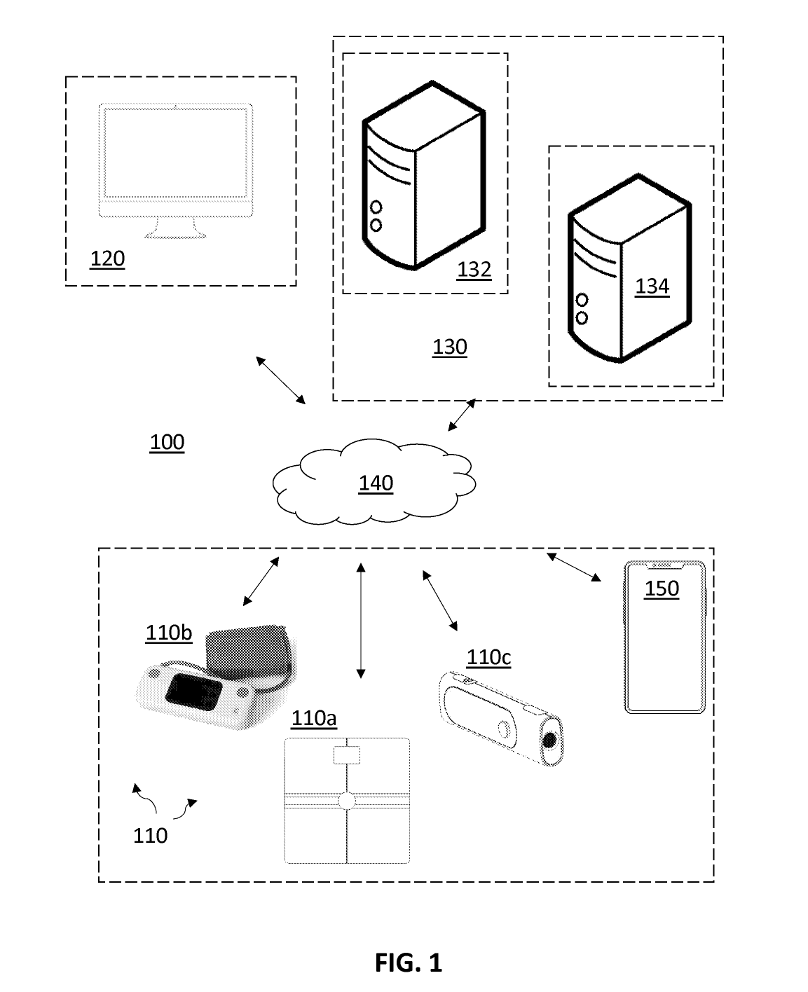

FIG. 1 illustrates a remote patient monitoring system 100, called RPM system 100. As used herein, the term “remote” in the expression “remote patient monitoring system” refers to the fact that the professional and the patient operate the respective components of the RPM system 100 from different physical locations, such that the professional does not need to be co-located with the patient or with the physiological measurement device during acquisition of physiological data. The term “remote” encompasses situations in which the professional is located in a separate room, building, facility, neighborhood, city, region, or any geographically distinct environment relative to the patient. It further encompasses implementations in which data is transmitted across local-area networks, wide-area networks, cellular networks, or the Internet, and does not require real-time synchronous communication between the patient and the professional. The term therefore excludes arrangements in which the professional directly observes or controls the measurement device at the time of measurement, and instead defines a technical architecture in which the physiological measurement device 110, the server 130, and the remote monitoring interface 120 operate cooperatively to allow the professional to review, interpret and act upon physiological data obtained at a distance.

The embodiment depicted in FIG. 1 is presented in simplified, schematic form for purposes of illustration and is not intended to be limiting with respect to the number, type, physical arrangement or implementation of the components shown, which may vary between different deployments of the RPM system 100. The RPM system includes at least one physiological measurement device 110, called device 110 (here a body scale 110a, a blood pressure monitor 110b, and/or a multiscope 110c (such as disclosed in EP24217550 or devices called BeamO™), but could comprise other devices, such as those disclosed in EP4386383 to Withings™, or devices U-Scan™ developed by Withings™), a remote monitoring interface 120, a server 130, which may comprise a first server 132 and a second server 134 and a communication network 140. In general, each physiological measurement device 110 may comprise one or more physiological sensors, a local user interface, a power supply (for example a battery or mains power adapter), and a communication module enabling the device 110 to exchange data with the communication network 140 either directly or via an intermediate device, and a plurality of distinct physiological measurement devices 110 may be associated with a single user account within the RPM system 100. The RPM system 100 may comprise also a user terminal 150, such as mobile device, like a smartphone, a smartwatch, or a laptop. In various embodiments, the mobile device 150 is configured to execute a patient-facing software application that implements a close monitoring interface for the user, to act as a gateway relaying data between the physiological measurement device 110 and the server 130, to locally store historical physiological measurements and contextual data, and/or to receive configuration information, alerts and software or firmware updates from the server 130 and forward them to the physiological measurement device 110. The mobile device 150 may, by way of non-limiting example, be implemented as a smartphone, a smartwatch, a tablet computer, a laptop computer or any other portable electronic device including a processor, a memory and a wired and/or wireless communication interface. The remote monitoring interface 120 is configured to run on a remote monitoring device (not shown on FIG. 1), which is remotely arranged from the device 110. The remote monitoring interface 120 may be implemented as software instructions executed by the remote monitoring device and may take the form of a native application, a web application, a web portal or another graphical user interface configured to receive, organize, filter, display and allow interaction with physiological data and contextual data transmitted from one or more physiological measurement devices 110. The remote monitoring device may, for example, be a desktop computer, a laptop computer, a tablet computer or another professional-grade computing device used within a clinic, hospital or telemedicine facility. The communication network 140 schematically represents any suitable wired and/or wireless communication infrastructure coupling the physiological measurement device 110, the remote monitoring device and the server 130, and may include, without limitation, one or more local‑area networks (LANs), wide‑area networks (WANs), cellular networks, the Internet, and/or short‑range wireless links such as Wi‑Fi® or Bluetooth®, arranged in any appropriate topology.

The server 130 is interfacing between the device 110 and the remote monitoring interface 120.

In a one-server embodiment, the server 130 is a single functional entity. In that case, the device 110, the server 130 and the remote monitoring interface 120 may be provided by a same provider.

In a dual-server embodiment, the server 130 includes the first server 132 which is interfacing between the second server 134 and the device 110, while the second server 134 is interfacing between the first server 132 and the remote monitoring interface 120. In that case, the device 110 and the first server 132 are generally provided by a first provider, while the second server 134 and the remote monitoring interface are provided by at least one second provider. The first server 132 and the second server 134 may communicate data through one or more API (application programming interface) for example. The first server 132 receives all data coming from the device 110 and sends all of them or a part of them to the second server 134. The remote monitoring interface 120 may only access data from the second server 134. In the dual server embodiment, the actions of the present description may be carried out by the first server 132 or the second server 134.

The RPM system 100 is used by various people, who are defined as follows: the user of the device 110 is referred to as a patient and the user of the monitoring device 120 is referred to as a professional, since he or she is a healthcare professional, such as a nurse or a physician. For clarity, in the context of the present description the terms “patient” and “user” may be used interchangeably to designate the individual whose physiological data are being measured and monitored, regardless of whether that individual is acutely ill, chronically ill or simply under preventive or routine follow-up care, and the term “professional” is intended broadly to encompass any authorized healthcare provider or member of a care team who is permitted to consult and/or act upon the monitored data, such as physicians, nurses, nurse practitioners, physician assistants, pharmacists, medical technicians and other allied health professionals. The professional and the user in a RPM system are usually not in the same room, but usually at their respective work or living locations, except for a specific appointment, such as a follow checkup. In typical home-monitoring scenarios, the patient operates the device 110 from a residence, workplace or assisted- living facility, while the professional accesses the remote monitoring interface 120 from a clinic, hospital, telemedicine center or other professional environment, so that physiological measurements and associated contextual data can be obtained by the patient at one time and location and reviewed or acted upon by the professional at a different time and/or location. Although FIG. 1 is described, for ease of explanation, with reference to a single patient and a single professional, the RPM system 100 can be configured such that a given professional monitors physiological data associated with a plurality of user accounts, each corresponding to a distinct patient enrolled in a remote patient-monitoring program. In some embodiments, the RPM system 100 may also be accessed in a limited and controlled manner by additional users, such as technical administrators responsible for maintaining the system or configuring user accounts, while preserving strict segregation between technical administration functions and medical decision-making functions performed by the professional.

In a typical situation, the device 110 generates physiological data from the patient during a measurement session initiated by the user or automatically scheduled by the RPM system 100 and sends the physiological data to the server 130 via the communication network 140, optionally after temporarily storing the physiological data and associated metadata (such as a timestamp, a user account identifier, and/or an identifier of the monitor 110) in an internal memory of the device 110. The physiological data may be transmitted in raw form (for example, systolic and diastolic pressure values and, where available, heart-rate values) and/or in processed form (for example, averaged values over several inflations, quality indicators or flags indicative of artefacts), using any suitable wired and/or wireless communication protocol supported by the communication network 140 and, in some embodiments, in an encrypted or otherwise secured format suitable for medical data. Then the remote monitoring interface may access the physiological data from the server 130 (or any data obtained from the physiological) via the communication network 140.

The RPM system 100 is aimed at gathering physiological data on the patient, via the device 110, and at providing them to the professional, via the remote monitoring interface 120. More specifically, the RPM system 100 is designed to establish a continuous or semi-continuous flow of clinically relevant information from the patient’s environment to the professional’s remote monitoring interface, thereby enabling timely detection of abnormal physiological trends and facilitating efficient triage of patients under remote care. The monitor 110 serves as the primary physiological measurement device for obtaining physiological data, which may include systolic and diastolic values, heart-rate measurements, quality indicators and any associated contextual data generated during the same measurement session. Once obtained, these data are transmitted over the communication network 140 to the server 130, where they may be stored, processed, enriched with metadata, and linked to a corresponding user account. The remote monitoring interface 120 may then retrieve the physiological data—optionally in enriched form or accompanied by an attributed notification level—and presents them to the professional in a structured and clinically actionable display mode. In this manner, the RPM system 100 not only transfers raw physiological measurements but also provides the professional with enhanced situational awareness regarding the circumstances under which each measurement was taken, ultimately improving the quality and efficiency of remote therapeutic decision-making.

The remote monitoring interface 120, the monitoring device 110 and the server 130 (including the first server 132 and the second server 134 if applicable) all exchange data using the communication network 140, which may be a hybrid network (ethernet, Wi-Fi, 3G, 4G, 5G, etc.).

### The device 110

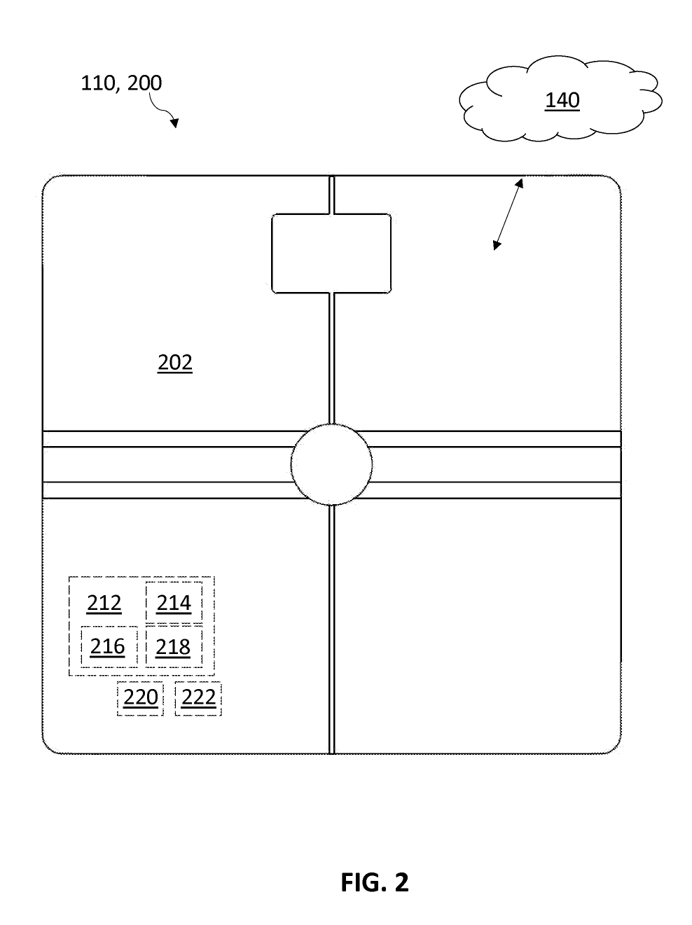

FIG. 2 illustrates a device 200 corresponding to the body scale 110a of FIG. 1. The device 200 includes at least one physiological sensor assembly 202, such as a weight sensor, a ECG (electrocardiogram) sensor, a PPG (photoplethysmogram) sensor, an IPG (impedanceplethysmogram) sensor, etc.

The device 200 notably generates physiological data using the physiological assembly 202.

As schematically shown on FIG. 2, the device 200 includes a control unit 212, comprising a processor 214, a memory 216 and a I/O (in/out) interface 218. The memory 216, which includes storage, is configured to store instructions, which, when executed by the processor 214, allows the device 200 to notably generate physiological data of a user, and send the physiological data . The I/O interface 218 manages the interactions of the control unit 212 with the other components. A communication module 220 enables the device 200 to exchange data, typically in a wireless manner with the communications network 140. A battery 222 stores energy to allow the device 200 to work without being electrically plugged.

The device 200 may store a history of the last measurement sessions, so that it can display information related to the evolution of physiological data.

In an embodiment the communication module 220 is a cellular module capable of exchanging data on a cellular network of the communication network, so that no user terminal 150 is required to forward any data generated by the device 200.

In an embodiment, the device 200 is multi-user, meaning that it may be shared by several users. To that end, each user has a user profile. Data generated for a same user is associated with the same user profile. The device 200 may therefore be configured to execute a local user-recognition process—such as by analyzing weight patterns, impedance signatures, or other physiological or behavioral parameters—to determine which registered user is likely operating the device during a given measurement session. In that case, a user recognition may be carried out by the device 200. However, in case of a misrecognition, the data are sent to the wrong registered user on the server 130 and will populate the wrong user profile with alien physiological data. Because this misattribution can propagate through server-side data logs, longitudinal trend analyses, and remote patient monitoring dashboards, even a single misclassification may distort the professional’s clinical understanding of the patient’s condition. In an RPM context—where therapeutic decisions, alerts, and care-plan adjustments may be triggered automatically or semi-automatically based on recognized physiological trends—such errors create a substantial risk of inappropriate clinical intervention or failure to detect genuine patient deterioration. This pitfall is paramount to avoid in an RPM system since medical actions may then be taken by the professional based on the physiological data.

In another embodiment, the device 200 is mono-user, meaning that any data generated by the device 200 is attributed to a unique user profile, regardless of who actually used the device 200. In this configuration, the device does not perform a local user-recognition process and therefore systematically forwards all generated physiological and/or recognition data to the server under the assumption that the registered user is the individual who performed the measurement. This configuration creates difficulties since another person may use the device 200 and the generated data will be attributed to the unique user profile and thus populate the user profile with alien physiological data. Such erroneous attribution can significantly degrade the integrity of the user profile over time, particularly in an RPM environment in which physiological trends are clinically monitored and may serve as the basis for medical decision-making. Accordingly, the inadvertent inclusion of “alien” data poses a substantial risk of misleading the healthcare professional, potentially triggering unnecessary clinical interventions, masking true patient deterioration, or otherwise compromising the reliability and safety of the RPM system. The embodiments disclosed herein therefore provide mechanisms—implemented at the server, the device, or both—to detect, mitigate, and remediate these risks by computing a recognition score and managing data attribution accordingly. This pitfall is paramount to avoid in an RPM system since medical actions may then be taken by the professional based on the physiological data.

When the user uses the device 110, 200, he or she starts a measurement session, during which physiological data of the user are to be generated, as well as recognition data. Therefore, unless said otherwise, any recognition data are generated along physiological data and are associated with them.

The physiological measurement device 110 is an electronic device.

### The remote monitoring interface 120

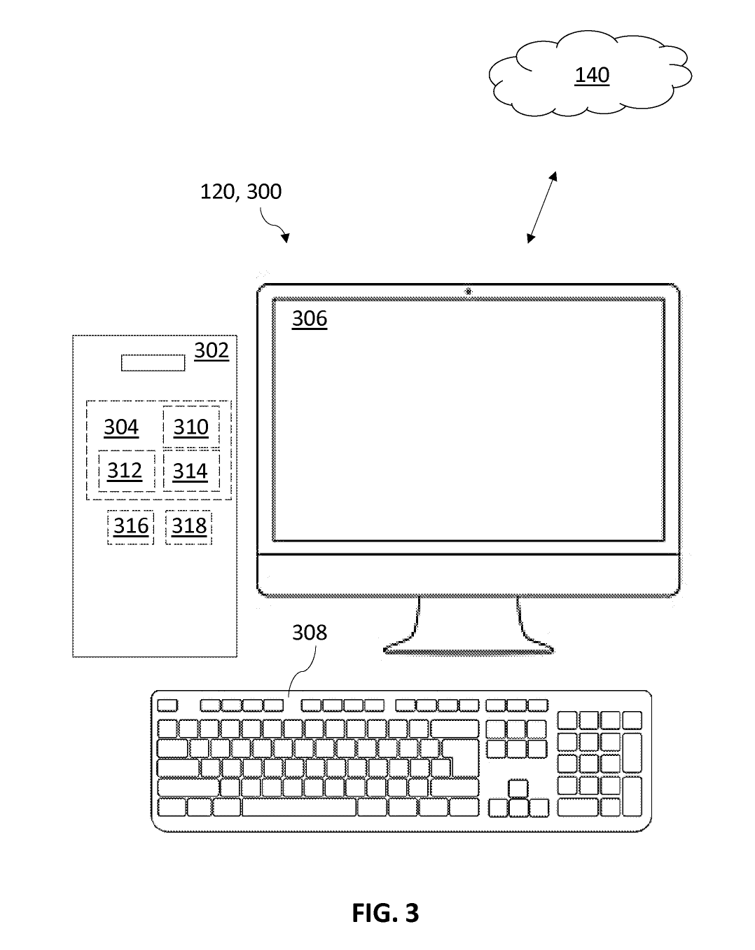

FIG. 3 illustrates a remote monitoring interface 300 corresponding to the remote monitoring interface 120 of FIG. 1. The remote monitoring device interface 300 can be run by a monitoring device 302 using a control unit 304 capable of processing data, including receiving data from the server 130 and displaying data. The remote monitoring interface 300 is configured for permitting an interaction with the professional. To that end the remote monitoring interface 300 may include a display 306, such as a screen, and a hardware input 308, such as a keyboard and/or a mouse, or a trackpad. The remote monitoring device 302 is typically a personal computer (e.g., a laptop).

The remote monitoring interface 300 is typically a software-based interface, which can be deployed as an app, for example accessible on an Appstore™, or a webapp or a web page accessible from a navigator. The remote monitoring interface 300 is generally provided as a SaaS (software as a service).

As schematically shown on FIG. 3, the control unit 304 comprises a processor 310, a memory 312 and an I/O (in/out) interface 314. The memory 312, which includes storage, is configured to store instructions, which, when executed by the process, allows the monitoring device 302 to notably display information on the display 306 and receive user input via the hardware input 308. The I/O interface 314 manages the interactions of the control unit 302 with the other components. A communication module 316 enables the monitoring device 302 to exchange data with the communication network 140. A battery 318 may store energy to allow the monitoring device to work without being electrically plugged.

To qualify as a RPM system 100, the remote monitoring interface 120, 300 is generally not activated on the device 110 or the user terminal 140 of the user, but only on the monitoring device 302 of the professional. In particular, the remote monitoring interface 120 is intentionally restricted to devices operated by authorized healthcare professionals so as to preserve a clear functional distinction between patient-facing components of the RPM system and professional-facing components. By contrast, the device 110 and any mobile device 150 associated with the user typically execute only patient-oriented interfaces, such as simplified measurement displays or close-monitoring interfaces, and do not provide access to the advanced monitoring dashboard, analytics tools, or multi-patient management capabilities available through the professional’s remote monitoring device 302.

The remote monitoring interface 120, 300 gives access to the professional to data from various user profiles, notably (and only) the user profiles of the users which are under a RPM program by the professional.

### The server 130

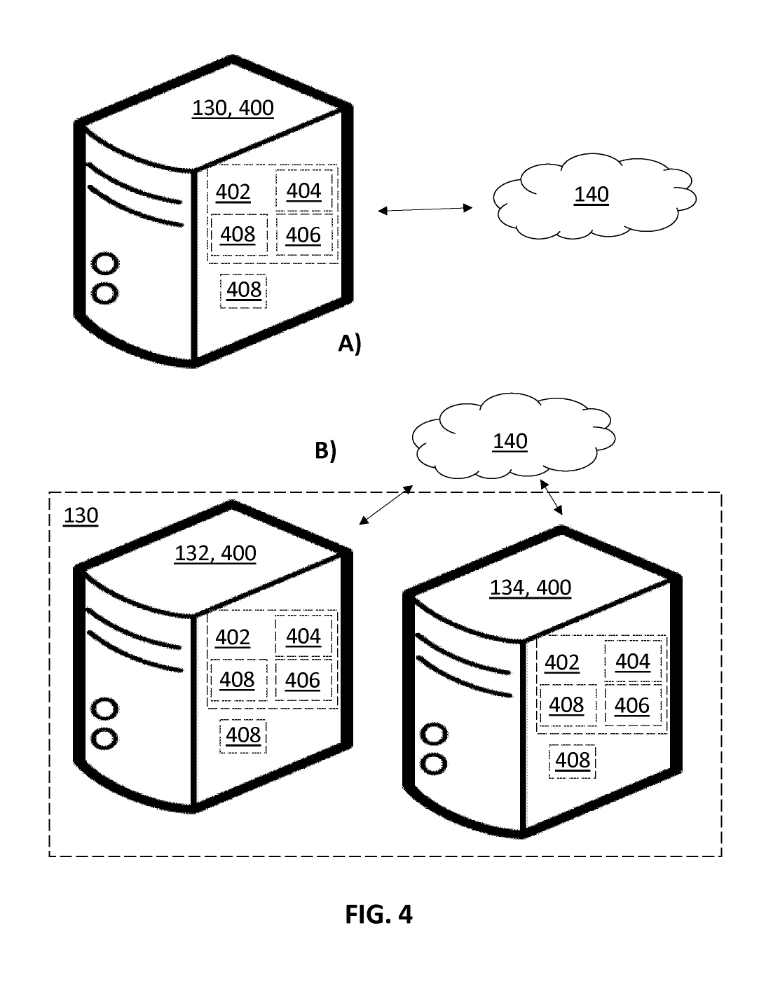

FIG. 4 illustrates two configurations A) and B), for the one-server embodiment and the dual-server embodiment. In configuration A), the server 130 of FIG. 1 includes a server 400. In configuration B), the server 130 includes a first server 132 and the second server 134 of FIG. 1, which each comprise a server 400.

The server(s) 400 are a remote hardware unit configured to receive, store and process data, for example received from the device 110 or from another server 400, and to send the physiological data or any related data to the remote monitoring interface 120 or to another server 400. Conversely, the server 400 is also configured to receive, store and process data received from the remote monitoring interface 120 and to send the data or any related data. The server 130 is typically remotely located from the device 110 and the remote monitoring interface 120. The server 130 may be located in a datacenter, for example a datacenter provided by a datacenter provider or by the device provided or by the hospital data center of the professional.

The server 400 store user profiles (with all the associated data) in a centralized manner and acts as a cloud server. In configuration B), the first server 132 may store a first user profile and the second server 134 may store a second user profile. Those profiles may be synchronized from time to time. The second user profile may be included in the first user profile.

As schematically shown on FIG. 4, any server 400 includes a control unit 402, comprising a processor 404, a memory 406 and a I/O (in/out) interface 408. The memory 406, which includes storage, is configured to store instructions, which, when executed by the process, allows the server to perform method as disclosed in the description. The I/O interface 408 manages the interactions of the control unit 402 with the other components. A communication module 408 enables the device 200 to exchange data, typically in a wired manner with the communications network 140.

Each of the servers 400 may technically include a plurality of servers.

In the rest of the description, the repartition of task between the first server 132 and the second server 134 will not be detailed and they will be referred to as the server 130. A specific section at the end deals with the repartition of action.

### General presentation

In the rest of the description, it will be considered that the device 110 is mono-user, with the user having a user profile in the server 130 or that the device 110 is multi-user and one of the users has a user profile in the server 130 (other users may have their own user profiles).

The device 110 is configured to generate physiological data during a measurement session, as described above. Typically, the physiological data include weight data. Additionally, they include impedance data (such as the fat mass, water mass, bone mass, etc.), ECG data, PPG data, etc. The physiological data have a timestamp, which corresponds to the time at which the physiological data were generated. The physiological data are representative of a state of the user and have a medical or wellness purpose. Therefore the physiological are data of interest for RPM.

The device 110 is also configured to generate recognition data during the same measurement session. The purpose of the recognition data is to determine whether the physiological data were generated by the user of the user profile of the server 130. The acquisition of recognition data should not be demanding or challenging for the user, which is why they are acquired by the device 110. As the recognition data are generated during a measurement session for generating physiological data, their generation does not request an additional input or effort from the user.

In an embodiment, the recognition data may be taken from the physiological data. For example, the physiological data include weight data (such as the weight of the user) and the recognition include or are the weight data (such as the weight of the user as well). For example, the physiological data may include impedance data and the recognition data include impedance data, with or without weight data. Other physiological data may be used, such as the ECG and the recognition data may include the ECG.

Alternatively, the recognition data are distinct from the physiological data. For example, the recognition data are weight data which does not represent the weight of the user, such as a weight evolution representing a weigh pattern at the scale by the user (leaning to the left or right, or stepping with the left foot rather than the right foot).

The server 130 receives the generated recognition data and the generated physiological data, from the measurement session, which are attributed to the user profile, either because the device 110 is mono-user or because the user profile was recognized on the device 110.

The server 130, using at least the recognition data, computes a recognition score representative of a likelihood that the user associated with the user account of the server 130 is at the origin of the measurement session, and thus of the physiological data and the recognition data. It will be appreciated that the recognition score is not an arbitrary numerical value or an abstract data manipulation, but rather a specifically engineered metric derived from concrete physiological inputs (e.g., weight, impedance, ECG parameters) and, in certain embodiments, time-dependent reference data stored in the user profile. The score is computed according to predetermined comparison functions that quantify physiological consistency, temporal evolution, and deviation thresholds. These functions are expressly designed to address known shortcomings of conventional multi-user or mono-user RPM systems, which often lack any technical mechanism for verifying whether a generated dataset truly originates from the intended patient. As such, the recognition score constitutes a technological improvement to remote patient monitoring systems by providing an automated, data-driven safeguard against misattribution of physiological data, thereby improving system reliability and reducing the risk of clinically dangerous errors. This recognition score is then used to manage the generated physiological data, for various purposes, either by the server 130 (this may differ between the one-server embodiment and the dual-server embodiment). Managing may involve attaching the recognition score to the physiological data and sending both of them, deleting the physiological data at the user profile, or generating instructions to delete the physiological data at the user profile or on the device 110. This will be disclosed in more detail later.

### The recognition score

In an embodiment, computing the recognition score is made using reference data, which are stored in the user profile at the server 130. The reference data allows to recognize the user based on the reference data.

The reference data may typically be previously generated recognition data, either the latest generated recognition data or a history of latest generated recognition data, so as to anticipate a trend. As it will be described later, the reference data may be updated with the generated recognition data. This allows the reference data to be up-to-date. This will be described in further detail later.

More precisely, computing the recognition score involves comparing the reference data and the recognition data. This comparison may involve comparing values of the reference data and of the recognition data. However, as recognition data may vary over time, especially when the recognition data are taken from the generated physiological data, the comparison may also involve comparing timestamps of the generated recognition data and of the reference data. In one non-limiting example, when the recognition data include the user’s weight, the server 130 may compute (i) a value difference Δv between the generated weight and the stored reference weight, and (ii) a time difference Δt between the timestamps of the two measurements. A recognition score may then be obtained by applying a predefined function f(Δv, Δt), such as a weighted combination or a rule set. This type of computation is only one example; more complex mathematical models, including decaying functions, logistic curves, or machine-learned evaluators trained on historical intra-user variation patterns, may also be applied. In another embodiment, the reference data also has a reference score attached thereto and the computed recognition score takes into account the recognition score of the reference data (notably it is calculated from the recognition score).

The computed recognition score is then compared to at least one threshold. In particular, a certainty threshold Ct is defined, which characterizes the fact that for a recognition score above the certainty threshold, it is assumed that the user of the user profile did take the measurement. In a similar manner, an uncertainty threshold Ut may be defined, which characterizes the fact that for a recognition score below the uncertainty threshold, it is assumed that the user of the user profile did not take the measurement. The thresholds Ct and Ut may be predetermined, dynamically learned, or adaptively updated based on usage patterns of the device 200 or on physiological variability expected for the particular user population. For example, Ct may be set to a value corresponding to the minimum recognition score historically achieved by confirmed measurements for that user, optionally adjusted by a safety margin to accommodate natural physiological fluctuation. Likewise, Ut may be established by analyzing distributions of known “non-user” measurements (e.g., test data, clinical datasets, or synthetic adversarial measurements) and selecting a cutoff below which the likelihood of authentic user attribution falls beneath an acceptable probability level. In another embodiment, Ct and Ut may be computed as statistical confidence bounds (such as one- or two-standard-deviation offsets) on the reference data or on long-term recognition-score trends, or may be derived from population-level models that estimate expected day-to-day or week-to-week physiological deviations. The thresholds may also be periodically re-evaluated or automatically tuned by the server 130 to reflect long-term changes in the user’s physiology or measurement behavior. More details will be given later.

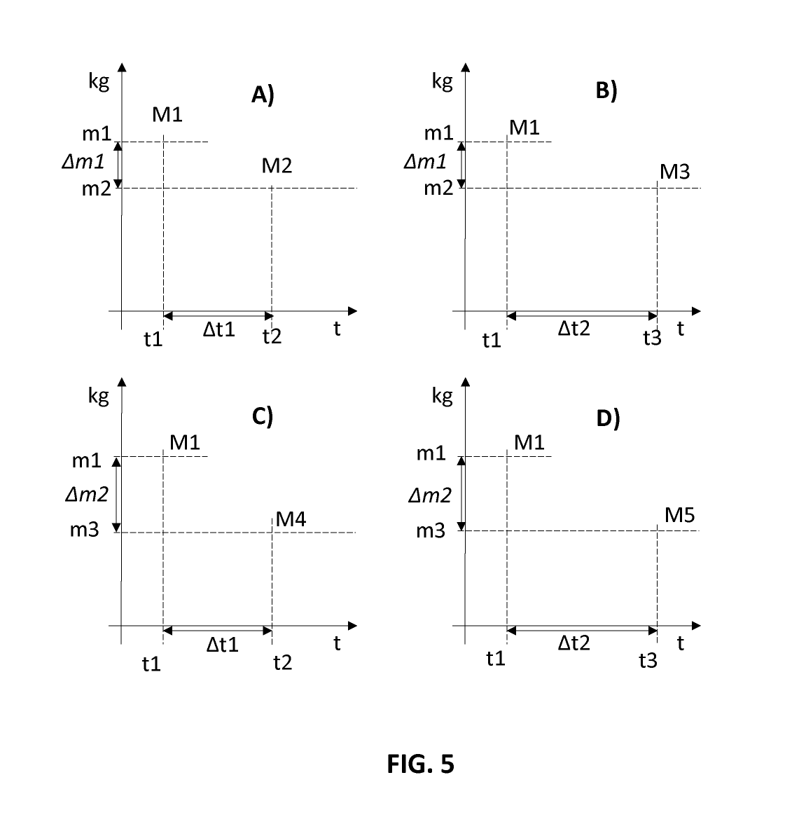

FIG. 5 illustrates different examples A), B), C) and D) in which the recognition data are weight data and more precisely the weight of the user of the user profile and the device 110 is a body scale.

The A), B), C) and D) examples illustrate four different situations. In all of them, measurement M1 represents the weight m1 (which is the value of the weight data) of a user at time t1 (which is the timestamp of weight data). The reference data were set to be m1. There are three situations: in situation A), at t2, a user steps on the body scale 110a for a measurement M2 of value m2, in situation B), at t3, a user steps on the body scale 110a for a measurement M3 of value m2, C), at t2, a user steps on the body scale 110a for a measurement M4 of value m3, D), at t4, a user steps on the body scale 110a for a measurement M5 of value m3.

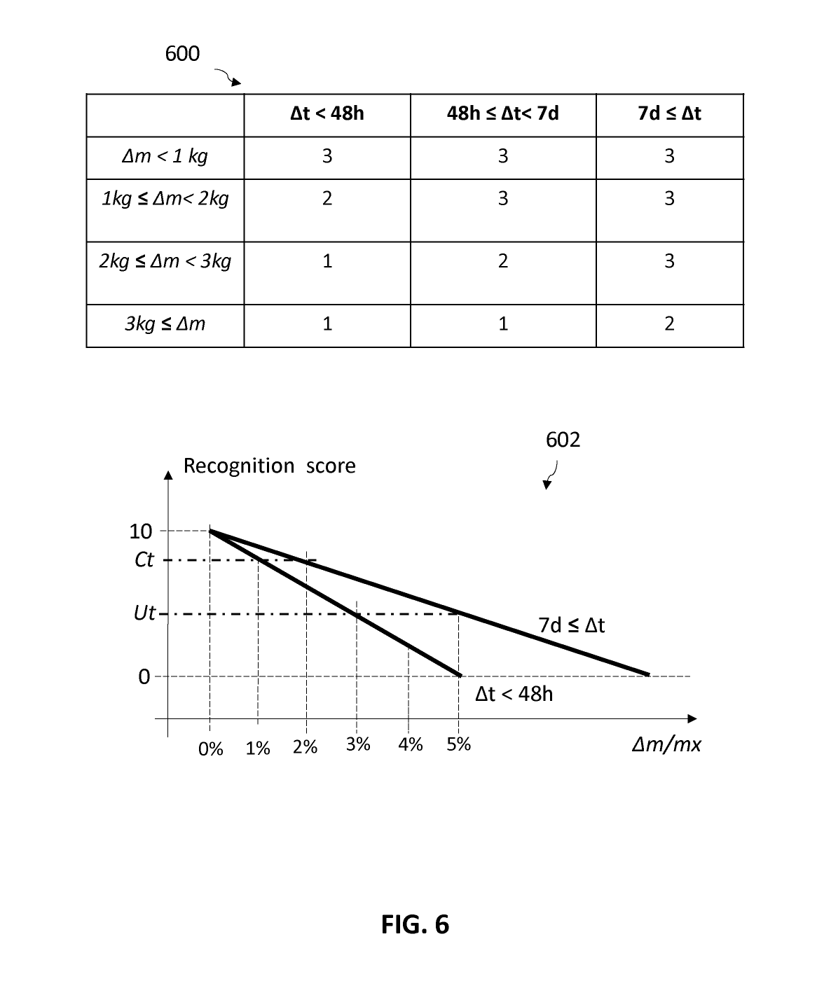

Timestamp t3 is later in time than t2 and m3 is lower than m2 in kg (represented by Δm1, Δm2, Δt1, Δt2 on FIG. 6).

Assuming that M1 was generated by the user of the user profile. Then the recognition score is defined so that, for a specific time difference Δt, the smaller the weight difference, the higher the recognition score, and the shorter the time difference the higher the score. Typically, thresholds are set up for several ranges of time differences. We call “function” the mathematical relation between the inputs (reference data, recognition data, and if applicable, the reference score) and the computed recognition score.

Table 600 on FIG. 6 illustrates a basic example of a function which provides values for recognition score, based on different value differences (Δm) and time differences (Δt). In this example, the recognition score may take three values (1, 2, 3). Instead of absolute value differences, the table 600 could use relative value differences (by computing Δm/mx, where mx is the value of the generated weight). The certainty threshold Ct may be here 2.5 and the uncertainty threshold may be 1.5.

The recognition score may be computed in a more refined way, with more values. For example, instead of thresholds as in table 600, the function defining the recognition score may be continuous function of Δm, wherein the function itself depends on Δt. Let mx be the weight of the measurement session.

Graph 602 on FIG. 6 shows a more refined example as described previously. Here the function is a linear function, whose slope depends on Δt. The slope defines the recognition score in function of Δm. Two slopes are given, for Δt < 48h (measurements close to each other in time) and for 7d ≤ Δt (d standing for days) (measurements spaced from each other). The certainty threshold Ct is reached for small weight variation for close measurements in time than for more spaced apart measurement in time (here 1% v. 2%). The uncertainty threshold Ut here is 3% for close measurements in time while it is 5% for more spaced apart measurement in time. The recognition score may take eleven values (from 0 to 10).

In another embodiment, the recognition score is computed based on the recognition score of the reference data (called reference score). For example, instead of having table 600 or the graph 700 which computes the recognition score in absolute, the function may provide an amount to subtract to the reference score, so as to compute the recognition score. In this case, the smallest amount to subtract is not zero, so that even recognition data identical to the reference score are not granted the highest recognition score. This way, even regular recognition data variations (e.g., small weight variations) will still lead the recognition score to go below the certainty threshold Ct at some point in time.

In this embodiment, the inputs of the function are the reference data, the recognition data, and the reference score and the output is the computed recognition score.

The function may be even more complex and involve for example a machine learning algorithm, which takes into account a history of recognition data (values and timestamps) to generate a more precise recognition score

### Actions to be taken in function of the recognition score

The server 130 stores the reference data, receives the generated physiological data and the generated recognition data, computes the recognition score. Then based on the recognition score, the server 130 takes different actions. The actions to be taken using the recognition score depends on the overall architecture of the RPM system. Some actions are carried out by the server 130, some by the remote interface monitoring 120 and some by the device 110. In the dual server architecture, the actions may be carried out by the first sever 132 and/or the second server 134.

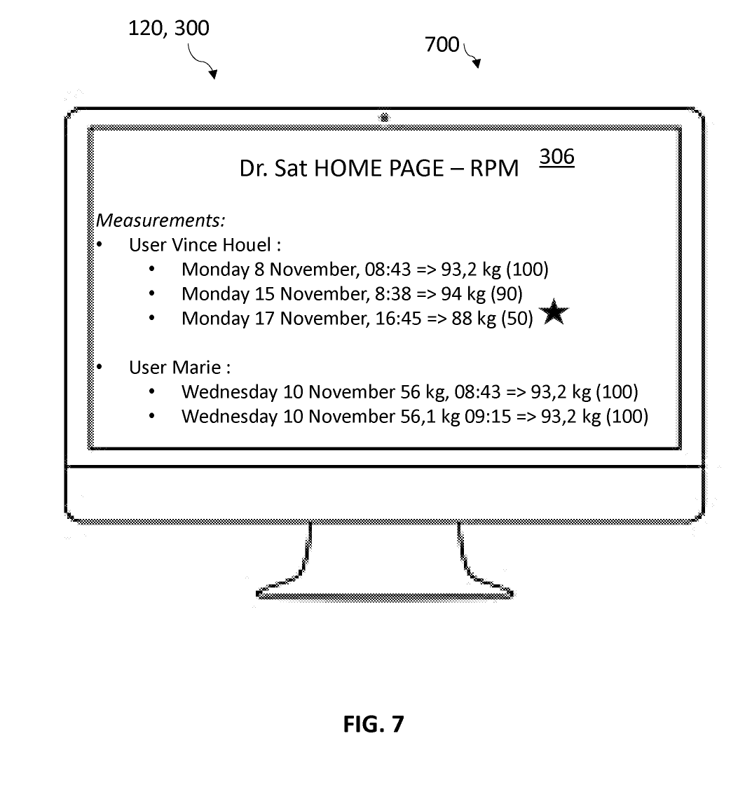

In an embodiment, the server 130 attaches the recognition score to the physiological data and the remote monitoring interface 120 displays the physiological data taking into account the recognition score. For example, a low recognition score (lower than the certainty threshold Ct or lower than the uncertainty threshold Ut) triggers an alert on the physiological data for the professional. FIG. 7 shows an interface 700 on the remote monitoring interface 120, with a specific visual feature for the physiological data with a low recognition score, such as lower than the certainty threshold Ct (here a star, for the weight measurement of Monday 17 November of user profile Vince Houel, followed by Dr. Sat on the remote monitoring interface 120 – all names and data are made up for the illustration), although the recognition score is not displayed as such on this example.

The server 130 stores the certainty threshold Ct. In an embodiment, in response to determining that the recognition score is above the certainty threshold Ct, the server 130 updates the reference data with the generated recognition data. As the physiological data were already attributed to the user profile, the server 130 does nothing in particular about them, so as to actually keep them in the user profile. For example, in relation with table 600 of FIG. 6, the certainty threshold Ct is equal to 2.5.

Additionally, or alternatively, the RPM system, in some situations that require clarity, may trigger a user request. The goal of the RPM system is to streamline the measurement process and to avoid any pain point. Any wrong data at the remote monitoring interface is an issue for the professional and the user of the user profile. Any interaction with the user which is not a medical interaction becomes a pain point which may disengage the user. The recognition score is aimed at finding a good balance between these two pain points.

The user profile at server 130 may store a notification channel, which allows the server 130 to communicate with the user. For example, the notification channel may exchange data with the user terminal 150 of the user. The notification channel may include a phone number, an app, etc.

In response to determining that the recognition score is below the certainty threshold Ct and for instance above the uncertainty threshold Ut (therefore between Ct and Ut),, the server may thus trigger a user request on the notification channel to confirm or reject that the measurement session was made by the user of the user profile, i.e. that the generated physiological data and the recognition data belongs to the user. This intermediate score range reflects a state of partial confidence in which the server 130 cannot reliably classify the measurement as authentic or non-authentic based solely on automated computation. Accordingly, the system escalates the decision to the user by initiating a verification request, thereby introducing a human-in-the-loop confirmation step that improves attribution accuracy without discarding potentially valid physiological data. The notification channel may include, for example, SMS messaging, push notifications through a dedicated application, automated voice calls, or secure in-app alerts, and the request may present the user with a simple confirmation prompt such as “Did you take a measurement at [timestamp]?” or an equivalent interface element. To be efficient, the request is to be sent a few minutes or hours after the measurement session so that the user can reliably recall whether he or she performed the measurement while avoiding unnecessary latency in RPM workflows. In certain embodiments, the server 130 may prioritize rapid notification when the physiological data indicate clinically significant values, or may batch notifications when multiple ambiguous measurements occur within a short time window.

The request is typically of the yes or no question type, such as: “was it you who took a measurement on [date and time]? ” (since the user terminal 150 is generally personal) or “was it [name of the user of the user profile] who took a measurement on [date and time]?”. FIG. 7 shows an example of a user request 700 on the user terminal 150, which is via a SMS on a phone.

When the notification channel comprises a phone number (which is almost systematically needed in RPM system, since the professional needs to be able to reach out to the user of the user profile), that the server 130 may send out an SMS to that phone number or make an automated phone call. The response may be a “YES” or a “NO” send through text or spoken over the phone. When the notification channel comprises an app, through which the server 130 may push data or questions, the user may directly respond in the app.

The response (confirmation or rejection) is then sent to the server 130 which then takes action accordingly.

If the response is a confirmation that the user who took the measurement is that of the user profile, then the reference data at the server 130 are updated with the generated recognition data. In particular, the confirmed recognition data become the new authoritative baseline against which future measurement sessions are evaluated, thereby improving the accuracy of subsequent recognition-score computations. The generated physiological data are kept at the user profile, since they were attributed on the server 130 to the user of the user profile. Depending on the embodiment, the recognition score (reference score) of the reference data is updated to the maximum possible, since there has been a confirmation that the recognition data belonged to the user of the user profile. Reinitializing the reference score to its maximum value ensures that future recognition-score calculations start from a state of high certainty, preventing premature triggering of verification requests and reducing unnecessary user burden. Additionally, this reset reestablishes the expected temporal decay or deviation-based reduction of the recognition score, thereby creating a predictable schedule by which the system may again seek user confirmation when sufficient divergence from the confirmed baseline naturally occurs. This resets the progressive decrease of the recognition score and thus create a regularly check via the notification channel. Such periodic revalidation, spaced according to the user’s physiological evolution, enhances long-term data integrity and maintains high confidence in the attribution of measurements within the RPM system.

If the response is a rejection that the that the user who took the measurement is that of the user profile, then the generated recognition data and the generated physiological data are removed from the user profile at the server 130. Additionally, the server 130 may send instructions to delete the latest measurement session stored on the device 110. This may be done by attributing a recognition score of the lowest possible value and having the removal performed in response to determining that the recognition score is under a deletion threshold or has the lowest possible value.

This user request may be triggered on the sole condition that the recognition score is below the certainty threshold Ct.

Conversely, the server 130 stores the uncertainty threshold Ut, and recognition score below Ut are assumed to mean that the user of the user profile did not made the measurement session, but it was someone else. Therefore, in an embodiment, in response to determining that the recognition score is below the uncertainty threshold Ut, the server 130 removes the generated recognition data and the generated recognition data from the user profile. The server 130 thus does not display the physiological data at the remote monitoring interface 120, thereby avoids the professional to spend time on wrong data.

However, because, for some patients susceptible of undergoing strong weight variations in a few day (cardiac compensation or decompensation for instance), it is necessary to monitor quick weight variations. In this embodiment, the physiological data are not deleted but a user request may be triggered as disclosed previously (hence the comparison with the certainty threshold Ct is sufficient).

In case the user does not respond to the user request within a predetermined time period (e.g. 3 days), the server 130 may consider that it is a confirmation (called confirmation by default), to avoid removal of data by default. However, this confirmation by default may be applied only when the recognition score is between the certainty threshold Ct and the uncertainty threshold Ut. Below the uncertainty threshold Ut, the server 130 may consider that an absence of response is a rejection (called rejection by default) and thus operate as disclosed above in case of a rejection.

In some embodiment, the user request is not systematically triggered but only after a plurality of measurement sessions for which the recognition score is under the certainty threshold. Upon confirmation, all the previous physiological data with a recognition score higher than the last one are also confirmed.

### The reference data

As explained previously, the reference data may be updated with the latest recognition data, since it is assumed that the most recent recognition data are more relevant than any previous reference data. In an embodiment disclosed above, the recognition score of the latest recognition data is attributed to the reference data.

In an embodiment, an initialization of the first reference data is performed, by manual input of reference data. This is particularly easy when the reference data is the weight of the user. For example, the RPM process may involve an on-boarding visit in person with the professional or any professional, so that the user may be weighed during the on-boarding visit. The professional then uploads the reference data on the server 130, for example via the remote patient monitoring interface 120.

In another embodiment, the initialization may involve an accompanied generation of reference data. This can take several forms.

In an example, the first measurement session on the device 110 is considered to be taken by the user of the user profile. Therefore the reference data will be taken from that first measurement session. It is fair to assume that, as the device 110 is first set up at the user’s home, the user of the user profile is the one setting up the device 110. In an example, the RPM system informs the user to generate reference data by using the device 110 during a certain time window, so that the RPM system knows it is the user of the user profile who generated the reference data.

In those cases, the reference data may be attributed the highest recognition score.

### Methods

The RPM system 100 disclosed above is configured to perform the following methods. Depending on the embodiments, some steps may be performed by the server 130 (one server embodiment) or any of the first server 132 and the second server 134 (dual server embodiment).

At 902, during a same measurement session, the device 110 generates physiological data and recognition data. The recognition data may be taken from the physiological data, to avoid adding constraint to the measurement session. For example the recognition data include weight data.

At 904, the device 110 sends the physiological data and the recognition data through the communication network 140. In a mono-user scale, those data are sent without condition on them to the server 130. This means that if an animal or a kin or a thief uses the device 110, the data will be attributed to the user profile on the server 130. The same applies if the device 110 carries out a wrong internal recognition process. At 906, the server 130 receives them.

At 906, the server 130 computes the recognition score, using the recognition data. The recognition is attached to the physiological data.

At 908, the server 130 determines whether the recognition score is above the certainty threshold Ct or below it. The server 130 may also determine whether the recognition score is above the uncertainty threshold Ut or below it. At 910, in response to determining that the recognition score is above the certainty threshold, the server 130 updates the reference data with the generated recognition data, and, if applicable, update the reference score with the computed recognition score. At 912, in response to determining that recognition score is below the certainty threshold, the server 130 trigger a user request on the notification channel of the user profile, to confirm or reject that the measurement session was generated by the user of the user account at the server. The notification channel uses for example the user terminal 150 of the user.

At 914, in response to receiving the confirmation, the server 130 updates the reference data of the user profile with the generated recognition data and, if applicable, the reference score is updated with the maximum possible value. At 916, in response to receiving the rejection or in response to determining that the recognition score is below the uncertainty threshold Ut, the server 130 removes the generated physiological data from the user profile. The recognition data 130, as they were attached to the generated physiological data, are also removed.

At 918, in response to receiving the rejection or in response to the removal, the server 130 sends instructions to the device 110 to remove the latest physiological data and recognition data of its memory. This makes sure that the user will not see a meaningless visual representation of his or her physiological data on a screen of the device 110.

In an embodiment, the server applies a rejection by default or a confirmation by default disclosed previously.

### One server or dual server configuration.

In a one server configuration for the server 130, all the steps are carried out by the single server 130, as defined previously.

In a dual server embodiment for the server 130, several variants exist.

In a variant, the first server 132 merely forwards data between the device 110 and the second server 132, so that the second server 132 performs most of the steps of the method performed by the server 130.

In a variant, the steps of server 130 which involve a communication with the device 110 are performed by the first server 132 and the other steps are performed by the second server 134. This involves transmission and reception steps between servers that are implicit in the description. In this variant, the first server 132 merely forwards data between the device 110 and the second server 134.

In a variant, the first server 132 computes the score and forwards the physiological data with the score to the second server 134, which performs the rest of the steps. Notably, the second server 134 triggers the notification channel. The second server 134 sends the confirmation to the first server which then updates the reference data and, if applicable, the reference score. As disclosed previously, each server 132, 134 may store a user profile of the user. Synchronization may be done only for data that is used by each server. For example, if the second server includes nutrition data that are not needed by the first server, no synchronization on those data is performed.

In a variant, which does not correspond to the dual server embodiment, one of the servers only communicates with the other server to retrieve the recognition data and the reference data, in order to compute the recognition score and to send it to the other server.

In practice, as already mentioned, the recognition data is the user weight. Therefore, the probability score is attached to the physiological data itself.

### Alternative design

In another embodiment, the recognition score is computed by the physiological measurement device 110 itself, and send to the server 130. As it has been shown, the computation of the recognition score requires reference data. Those reference data may be received from the server or, as they may be previous recognition data, are already stored at the device 110, in a user profile on the device 110. Once received by the server 130, the method is similar to any disclosed in this description, notably the comparison with the thresholds, the user request, etc.

In an embodiment, there is provided a non-transitory computer-readable medium storing instructions which, when executed by one or more processors of a server of a remote patient monitoring, RPM, system, cause the server to:

receive, from a physiological measurement device, generated physiological data and generated recognition data associated with a user profile stored at the server,

compute a recognition score based at least on the generated recognition data and reference data stored in the user profile, the recognition score being representative of a likelihood that a user associated with the user profile generated the physiological data,

compare the recognition score to at least one threshold including a certainty threshold and optionally an uncertainty threshold,

when the recognition score is above the certainty threshold, maintain the physiological data as attributed to the user profile and update the reference data with the generated recognition data,

when the recognition score is below the uncertainty threshold, remove at least the generated physiological data from the user profile and optionally send an instruction to the physiological measurement device to delete the generated physiological data and the generated recognition data, and

when the recognition score is between the certainty threshold and the uncertainty threshold, trigger, via a notification channel associated with the user profile, a user request to confirm or reject that the physiological data and the recognition data are to be attributed to the user profile, and process a received confirmation or rejection to update or remove the physiological data and the recognition data from the user profile.

In an embodiment, the instructions further cause the server to provide, to a remote monitoring interface, only physiological data that has been confirmed or inferred as originating from the user based on the recognition score, such that the physiological data presented on the remote monitoring interface is usable by a healthcare professional to support a diagnostic assessment.

In certain embodiments, the techniques disclosed herein are implemented, in whole or in part, as a computer program product. The server 130 and/or the control unit 402 of each server 400 may comprise one or more processors 404 configured to execute program instructions stored on a non-transitory computer-readable medium. The program instructions, when executed, cause the server 130 to perform the steps described in the present disclosure, including, for example, receiving physiological data and recognition data from the device 110, 200, computing the recognition score based on the recognition data and reference data stored in a user profile, comparing the recognition score to one or more thresholds, and managing the generated physiological data in accordance with the computed recognition score.

The non-transitory computer-readable medium may include, by way of non-limiting example, semiconductor memory (such as ROM, RAM, flash memory, EEPROM), magnetic storage (such as hard disk drives or magnetic tapes), optical storage (such as CD-ROMs, DVDs, or Blu-ray discs), or any other physical storage medium capable of tangibly embodying computer-executable instructions. The program instructions may be provided as a single software module or as a plurality of cooperating modules distributed across the server 130, the first server 132, the second server 134, and, in certain embodiments, the device 110, 200.

When executed by the processor 404, the instructions may cause the server 130 to compute the recognition score as described herein, for example by comparing generated recognition data with reference data, determining value and time differences, and applying one or more predetermined functions to obtain a numerical score indicative of the likelihood that the measured physiological data originates from the user associated with the user profile. Additional instructions may cause the server 130 to compare the recognition score to the certainty threshold Ct and the uncertainty threshold Ut, to determine whether to (i) store and use the physiological data as authentic, (ii) delete or suppress the physiological data as non-authentic, or (iii) trigger a user request via the notification channel when the recognition score lies between Ct and Ut.

Further instructions may cause the server 130 to update the reference data with confirmed recognition data, to adjust or recalculate thresholds based on historical score distributions, to generate and transmit control commands to the device 110, 200 for deleting or retaining locally stored measurement sessions, and to provide filtered and verified physiological data to the remote monitoring interface 120 for presentation to a healthcare professional. In this way, the computer program product implements the disclosed method in a concrete RPM system, resulting in tangible modifications to the storage, transmission, and display of physiological data used for diagnostic and monitoring purposes.

In an embodiment, there is provided a remote patient monitoring, RPM, system comprising:

a physiological measurement device configured to:

generate physiological data during a measurement session, and

generate recognition data associated with the physiological data;

a server in data communication with the physiological measurement device, the server being configured to:

store a user profile associated with a user, the user profile including reference data and a notification channel associated with a user terminal,

receive, from the physiological measurement device, the physiological data and the recognition data, wherein the physiological data and the recognition data are attributed to the user profile,

compute, based at least on the recognition data and the reference data, a recognition score representative of a likelihood that the user associated with the user profile generated the physiological data,

compare the recognition score to at least a certainty threshold and an uncertainty threshold, the certainty threshold being higher than the uncertainty threshold, and

in response to the comparison, manage the physiological data by:

when the recognition score is above the certainty threshold,

maintaining the physiological data as stored in the user profile, and

updating the reference data with the recognition data;

when the recognition score is below the uncertainty threshold,

removing at least the physiological data from the user profile and optionally sending an instruction to the physiological measurement device to delete the physiological data and the recognition data from a memory of the physiological measurement device; and

when the recognition score is between the certainty threshold and the uncertainty threshold,

triggering, on the notification channel, a user request to confirm or reject attribution of the physiological data and the recognition data to the user profile, and

in response to a received confirmation, updating the reference data with the recognition data and maintaining the physiological data as stored in the user profile, and

in response to a received rejection, removing the physiological data and the recognition data from the user profile;

and a remote monitoring interface configured to receive, from the server, physiological data stored in the user profile and to display the physiological data to a healthcare professional for diagnostic or monitoring use.

In various embodiments, the systems and methods described herein provide a concrete, technical solution to the problem of misattributed physiological data in remote patient monitoring environments. Conventional RPM systems generally assume that all data transmitted from a given device originates from the intended patient, or rely on rudimentary, on-device user recognition that cannot be reliably audited or corrected at the server side. As a result, prior art systems are vulnerable to alien data being stored in a patient’s record when another person, an animal, or even a thief uses the device. The present invention improves the operation of RPM systems by introducing a specific server-implemented mechanism that computes a recognition score based on reference data and current recognition data, compares that score to one or more thresholds, and automatically manages physiological data in accordance with the computed score.

In particular, the recognition score is not an arbitrary numerical output but is generated by applying predetermined comparison functions to concrete, measurable physiological parameters and associated timestamps, as described earlier. The server 130 (and/or the device 110, 200 in certain embodiments) implements specific processing steps to calculate the recognition score, to determine whether the score exceeds a certainty threshold Ct or falls below an uncertainty threshold Ut, and to execute corresponding actions such as retaining, flagging, deleting, or seeking confirmation of the data. These actions are carried out by the server 130 within a concrete system architecture comprising the device 110, the communication network 140, the remote monitoring interface 120, and optionally dual servers 132, 134. The resulting system does not merely manipulate data in the abstract; instead, it alters the way physiological data are stored, displayed, and acted upon in a functioning RPM infrastructure.

The output of the method implemented by the system is directly tied to practical clinical use. For example, when the recognition score exceeds Ct, the corresponding physiological data are maintained in the user profile and made available through the remote monitoring interface 120 as part of the patient’s longitudinal record. The healthcare professional can rely on these data, with an indication (explicit or implicit) that they are highly likely to be authentic, to support diagnostic assessments such as evaluating weight gain associated with heart failure, monitoring blood pressure trends, or assessing changes in body composition. Conversely, when the recognition score falls below Ut, the system may automatically remove the data or prevent it from being displayed in the patient’s record, thereby preventing misleading information from influencing diagnostic conclusions. In intermediate cases (between Ct and Ut), the system initiates a confirmation workflow through the notification channel so that the professional ultimately receives a dataset whose attribution has been either algorithmically validated or explicitly confirmed by the user.

Accordingly, the invention provides technical benefits over prior art RPM systems that accept device-originated data without robust verification. By introducing the recognition score, reference data, dynamic thresholds, and automated data-management rules, the system improves the overall integrity and reliability of the physiological data used for clinical decision-making. This reduces the likelihood of false alarms, unnecessary therapeutic interventions, and missed detections of genuine patient deterioration that may arise from misattributed measurements. The invention therefore enhances not only the accuracy of the underlying data but also the safety and effectiveness of diagnostic and monitoring workflows built upon those data.

Moreover, the described mechanisms improve the functioning of the RPM system itself. The server-side processing offloads complex recognition logic from the device, allows centralized adjustment of thresholds and functions, and enables coordinated actions across multiple servers and interfaces (e.g., dual-server architectures with distinct providers). The interplay between the recognition score computation, the notification channel, and the remote monitoring interface 120 creates a feedback loop in which confirmed measurements update reference data and recalibrate the recognition process over time. This yields a self-correcting system that continually adapts to the user’s evolving physiology and behavior, providing an ongoing technical improvement in how RPM systems attribute, curate, and present physiological data for diagnostic use.

It will be appreciated that once the recognition score is computed, the system performs concrete, technical operations that directly affect how physiological data are stored, transmitted, displayed, and acted upon within the remote patient monitoring ecosystem. These actions are not abstract data processing steps but correspond to explicit control flows and system-level changes implemented by the server 130, the device 110/200, or both. For example, when the recognition score exceeds the certainty threshold Ct, the server 130 immediately classifies the data as authentic and stores it within the user's profile as clinically actionable physiological information. This storage step may also trigger downstream processes, such as updating trend charts on the remote monitoring interface 120, generating compliance metrics, or synchronizing updated baselines across multiple servers in a dual-server configuration. These actions materially alter how the physiological data is used and displayed within the system, providing the healthcare professional with reliable, attribution-confirmed data from which diagnostic assessments may be drawn.

When the recognition score falls below the uncertainty threshold Ut, the system initiates a different set of concrete steps. In such embodiments, the server 130 may delete the data from the user profile, suppress transmission to the remote monitoring interface 120, or tag the measurement as invalid for diagnostic use. The server may also generate a control instruction sent back to the device 110/200 to remove the local copy of the measurement session from device memory, thereby preventing the user from encountering misleading or non-authentic trends on the device itself. These operations ensure that alien data does not contaminate the patient’s longitudinal physiological record and that such erroneous data does not propagate through clinical dashboards, automated alert systems, or billing workflows.

In the intermediate case in which the recognition score lies between Ct and Ut, the system executes additional actions beyond mere classification. The server 130 automatically triggers the notification channel, causing a real-time communication to be sent to the user terminal 150. The initiation of this communication constitutes a direct system action tied to the computed score. Depending on the user’s response, the server 130 then performs a subsequent set of operations: (1) updating the reference data and reinforcing the authenticated physiological dataset, or (2) purging the data from the user profile and removing it from all downstream outputs. In embodiments where no user response is received within a predetermined time, the system may implement “confirmation by default” or “rejection by default” logic, which again results in concrete database modifications and can initiate device-side deletion commands. These actions collectively ensure that unverified data is never displayed or used in clinical workflows without undergoing a structured evaluation process.

Accordingly, the recognition score directly governs machine-implemented operations that materially modify the state of the RPM system, including database contents, device memory, network communications, and user-interface outputs. These system-level changes provide technical safeguards against misattribution and enable healthcare professionals to rely on the resulting physiological datasets for diagnostic interpretation, thereby tightly coupling the claimed method to a practical application and addressing a technical problem not solved in prior art RPM platforms.

In various embodiments, the method implemented by the RPM system further comprises one or more final actions that use the managed physiological data in a concrete clinical or operational workflow. For example, after the generated physiological data have been retained, updated, or removed based on the recognition score and the applicable thresholds, the server 130 may transmit the resulting, attribution-validated physiological data to the remote monitoring interface 120 for display to a healthcare professional. The remote monitoring interface 120 may update a trend graph, a tabular view, or a dashboard indicator associated with the user profile, thereby providing the professional with an accurate and current representation of the patient’s physiological status that can be relied upon for diagnostic and therapeutic decision-making.

In another embodiment, the method further comprises generating and transmitting a clinical alert or notification based on the validated physiological data. For instance, if the verified data indicate that a parameter such as body weight, blood pressure, or impedance-derived congestion index exceeds a predefined clinical threshold, the server 130 may automatically create an alert record and push it to the remote monitoring interface 120, send an electronic message to a clinical messaging system, or generate a task in a care-management platform. The alert may prompt the healthcare professional to review the patient’s status, schedule a teleconsultation, adjust a treatment plan, or order additional diagnostic tests. In this way, the method culminates in a concrete system action that directly influences patient management workflows, rather than ending at abstract data manipulation.

In yet another embodiment, the method includes exporting the validated physiological data, together with an indication of the recognition score or an attribution-confidence status, to an external electronic health record (EHR) system or hospital information system. The server 130 may format the data according to a standardized interchange protocol and transmit the data over a secure interface so that it becomes part of the patient’s official medical record. This export step ensures that only data that have passed the recognition-score based verification process are committed to the EHR, thereby improving the integrity of downstream clinical documentation, coding, and billing processes.

In some implementations, the final step of the method further comprises generating a summary report or stratification indicator for a cohort of patients based on the validated data. For example, the server 130 may compute, for a group of patients under an RPM program, adherence rates, average weight trends, or congestion-risk categories exclusively from measurements that have been authenticated by the recognition-score mechanism. The resulting report or indicator may be displayed on a population-level dashboard, printed, or transmitted to a care team to support resource allocation, triage decisions, and program evaluation. Thus, the method not only verifies data at the individual level but also produces tangible outputs used in real-world clinical and operational decision-making.

Through these additional final steps, the method implemented by the RPM system is explicitly tied to practical applications, including updating clinical user interfaces, triggering alerts, interfacing with EHR systems, and generating actionable reports. These concrete operations demonstrate that the invention is directed to improving the functioning of remote patient monitoring systems and the reliability of diagnostic and care-management workflows, rather than merely performing abstract data analysis.

It will be appreciated that the actions and functions performed by the disclosed system cannot be executed in the human mind, nor can they be carried out manually in any practical or reliable manner. The computation of the recognition score requires processing of multiple physiological measurements, timestamp correlations, deviation functions, and threshold comparisons across potentially thousands of data points transmitted over time. The server further executes automated management steps—such as selectively updating reference data, deleting physiological datasets, issuing device-side deletion commands, triggering real-time notification events, and synchronizing results across single-server or dual-server architectures—that necessarily involve machine-implemented operations, memory manipulation, wireless communication protocols, and coordinated control of distributed computing components. These functions depend on rapid numerical evaluation, secure data exchange, and autonomous workflow execution that cannot be replicated or meaningfully approximated by human cognition. Accordingly, the invention provides a technological solution implemented through specific hardware and software components, rather than a mental process, thereby reinforcing that the claimed subject matter is directed to patent-eligible practical applications rather than abstract ideas.

Expressions such as “comprise”, “include”, “incorporate”, “contain”, “is” and “have” are to be construed in a non-exclusive manner when interpreting the description and its associated claims, namely construed to allow for other items or components which are not explicitly defined also to be present. Reference to the singular is also to be construed in be a reference to the plural and vice versa.

The articles "a" and "an" may be employed in connection with various elements and components, processes or structures described herein. This is merely for convenience and to give a general sense of the compositions, processes or structures. Such a description includes "one or at least one" of the elements or components. Moreover, as used herein, the singular articles also include a description of a plurality of elements or components, unless it is apparent from a specific context that the plural is excluded.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

A person skilled in the art will readily appreciate that various features, elements, parameters disclosed in the description may be modified and that various embodiments disclosed may be combined without departing from the scope of the invention. For example, various aspects of the present disclosure may be used alone, in combination, or in a variety of arrangements not specifically described in the embodiments described in the foregoing and is therefore not limited in its application to the details and arrangement of components set forth in the foregoing description or illustrated in the drawings. For example, aspects described in one embodiment may be combined in any manner with aspects described in other embodiments.

Having described above several aspects of at least one embodiment, it is to be appreciated various alterations, modifications, and improvements will readily occur to those skilled in the art. Such alterations, modifications, and improvements are intended to be aspects of this disclosure. Accordingly, the foregoing description and drawings are by way of example only.

1. A remote patient monitoring, RPM, system comprising at least:
a server storing a user profile associated with a user, and
a physiological measurement device, wherein every data generated by the physiological measurement device is systematically associated with the user profile of the server,
wherein the RPM system is configured to:
generate physiological data by the physiological measurement device,
generate recognition data by the physiological measurement device, wherein the recognition data are associated with the physiological data,
receive by the server the generated recognition data and the generated physiological data, wherein the physiological data and the recognition data are attributed to the user profile of the server,
compute by the server a recognition score based at least one the generated recognition data, the recognition data being used to determine whether the physiological data were generated by the user of the user profile,
manage the generated physiological data based at least one the recognition score.

2. The RPM system of claim 1, wherein managing the physiological data includes at least one of: attaching the recognition score to the physiological data, removing the physiological data of the user profile based on the recognition score and/or trigger a user request to confirm or reject the physiological data, generating instructions to delete the physiological data of the user profile based on the recognition score.

3. The RPM system of claim 1 , wherein the user profile includes reference data, and the recognition score is computed based on a comparison between the reference data and the recognition data.

4. The RPM system of claim 3, wherein computing the recognition score takes into account:
a value difference between a value of the generated recognition data and a value of the reference data, and
a time difference between a timestamp of the generated recognition data and a timestamp of the reference data.

5. The RPM system of claim 3, wherein a reference score is attached to the reference data and computing the recognition score takes into account the reference score, so that the recognition score can only be strictly lower than the reference score

6. The RPM system of claim 1, further configured to:
in response to determining that the recognition score is above a certainty threshold, update the reference data with the generated recognition data.

7. The RPM system of claim 3, further comprising initializing the reference data of the user profile, wherein initializing is done by either one of:
sending through a notification channel a user request to use the physiological measurement device during a time window and receiving recognition data during that time window, and setting the reference data as the recognition data.
initially providing the reference data by a third party,
manually inputting the reference data.

8. The RPM system of claim 1, further configured to:
in response to determine that the recognition score is below an uncertainty threshold, remove the generated physiological data and the generated recognition data from the user profile.

9. The RPM system of claim 1, wherein the user profile includes a notification channel to the user, the system being further configured to:
in response to determining that the recognition score is below the certainty threshold, trigger by the server a request on the notification channel to confirm or reject that the received physiological data and the recognition data are to be attributed to the user profile.

10. The RPM system of claim 9, being further configured to:
in response to receiving the confirmation, update the reference data of the user profile with the generated recognition data, and/or
in response to receiving the rejection, remove the generated recognition data and the generated physiological data of the user profile.

11. The RPM system of claim 1, wherein the server includes a first server and a second server, and the user profile includes a first user profile stored at the first server and a second user profile at the second server, wherein the second user profile includes at least the first user profile, wherein the second user profile and the first user profile are associated with the same user, and the second server is connected to a remote monitoring interface, wherein the RPM system is further configured to:
send by the first server the physiological data with the recognition score to the second server,
remove the generated physiological data of the second user profile based on the recognition score.

12. The RPM system of claim 11, further configured to:
in response to determining that the recognition score is below an uncertainty threshold, remove the generated physiological data and the generated recognition data from the user profile,
wherein determining and removing are performed by the second server, and removing is done on the second user profile.

13. The RPM system of claim 11, wherein the user profile includes a notification channel to the user, the system being further configured to:
in response to determining that the recognition score is below the certainty threshold, trigger by the server a request on the notification channel to confirm or reject that the received physiological data and the recognition data are to be attributed to the user profile,
wherein the notification channel is included in the second user profile, and triggering is performed by the second server.

14. The RPM system of claim 1, further configured to:
send to the physiological measurement device instructions to delete the generated physiological data and the generated recognition data.

15. The RPM system of claim 1 further comprising a remote monitoring interface, wherein the remote monitoring interface has a specific visual feature for the physiological data with a recognition score under a certainty threshold.

16. The RPM system of claim 1, wherein the recognition data are taken from the physiological data.

17. The RPM system of claim 1, wherein the recognition data include at least one of weight data and impedance data.

18. A method of remotely monitoring patients, using the RPM system of claim 1, the method comprising:
generating physiological data by the physiological measurement device,
generating recognition data by the physiological measurement device, wherein the recognition data are associated with the physiological data,
receiving by the server the generated recognition data and the generated physiological data, wherein the physiological data and the recognition data are attributed to the user profile of the server,
computing a recognition score based at least one the generated recognition data, the recognition data being data used to determine whether the physiological data were generated by the user of the user profile,
managing the generated physiological data based at least one the recognition score.

19. A body scale storing a user profile associated with a user, and configured to exchange data with a server, wherein every data generated by the physiological measurement device is systematically associated with the user profile of the server, wherein the body scale is configured to:
generate physiological data,
generate recognition data, associated with the physiological data,
compute a recognition score based at least one the generated recognition data, the recognition data being data used to determine whether the physiological data were generated by the user of the user profile,
send the recognition score and the physiological data to server,
manage, at the device and/or the server, the generated physiological data based at least on the recognition score.

20. A method of remotely monitoring patients, using the body scale of claim 19, the method comprising:
generating physiological data,
generating recognition data, associated with the physiological data,
computing a recognition score based at least one the generated recognition data, the recognition data being data used to determine whether the physiological data were generated by the user of the user profile,
sending the recognition score and the physiological data to server,
managing, at the device and/or the server, the generated physiological data based at least on the recognition score.
